(19) United States
(12) Patent Application Publication
(10) Pub. No.: US 2023/0330217 A1
(43) Pub. Date: Oct. 19, 2023

(54) Herpes 1 Antibody System

(71) Applicant: Raymond Joel Rairie, Manchester, NY (US)

(72) Inventor: Raymond Joel Rairie, Manchester, NY (US)

(21) Appl. No.: 18/212,711

(22) Filed: Jun. 21, 2023

Related U.S. Application Data
(63) Continuation-in-part of application No. 17/063,706, filed on Oct. 5, 2020.
Application No. 17/063,706 is a continuation-in-part of application No. 15/663,786, filed on Jul. 30, 2017, now abandoned.

(51) Int. Cl. A61K 39/245; A61P 37/04

(52) U.S. Cl. CPC A61K 39/245; A61P 37/04; A61K 2039/55

(57) ABSTRACT

The PINBALL HERPES ANTIBODY SYSTEM prevents people from getting HERPES. It prevents people from getting HERPES SIMPLEX 1 VIRUS (Hsv-1) (HERPES 1) on their genitals, mouth, anus, hands, eyes and face. It also prevents people from getting SIMPLEX 2 VIRUS (Hsv-2) (HERPES 2) on their genitals, mouth, anus, hands, eyes and face. And it prevents a baby from getting infected with HERPES 1 and HERPES 2 during birth.

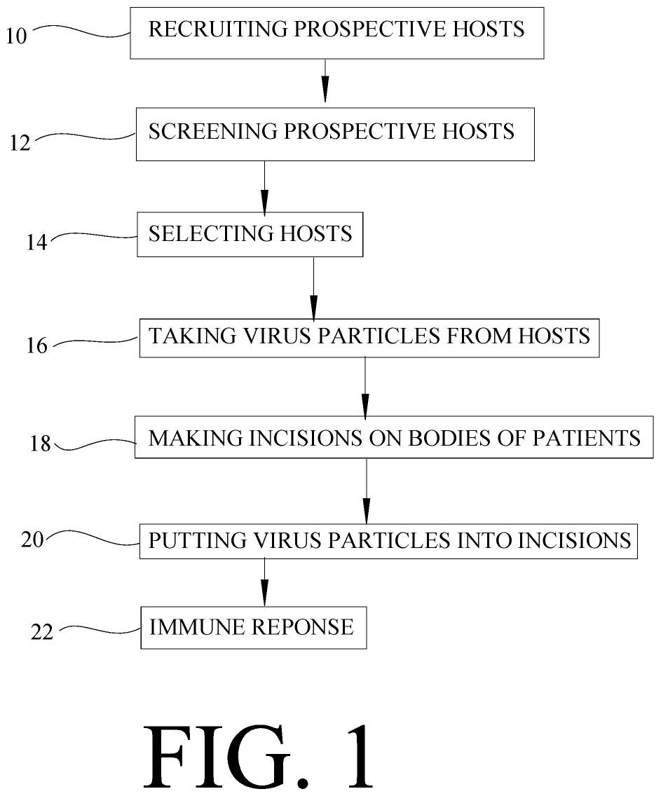

This application is a Continuation-In-Part of application Ser. No. 15/663,786, filed on Jul. 30, 2017, which is incorporated by reference.

### FIELD

This application is designed to: (1) generate a safe process that will protect people from getting infected with HERPES SIMPLEX 1 (HSV-1)—commonly known as “HERPES 1”—on or around their genitals, mouth, and anus. 100021 And (2) to protect people from getting infected with HERPES SIMPLEX 2 (HSV-2)—commonly known as “HERPES 2” or around their genitals, mouth, and anus. 100031 And (3) prevent babies from getting infected with HERPES 1 and/or HERPES 2 during birth (the baby could die if the baby gets infected). 100041 The PINBALL HERPES ANTIBODY SYSTEM involves the composition of matter.

The matter being antibodies for both HERPES 1 and HERPES 2.

### BACKGROUND

The purpose of this patent is to benefit humanity and society and civilization. People enjoy sex very much. Unfortunately—sometimes people contract Herpes on their private parts or mouth. Millions of people have Herpes on their private parts and mouth. Some people have a natural ability to fight off Herpes—this is good for them. Others are able to take medication to control outbreaks. Unfortunately, Herpes medication does not always work—or only works to a certain point. Unfortunately, some people have painful outbreaks on their private areas and mouth two or three or more times per year—for all their life. They lose work. And (unfortunately) sometimes (unintentionally) spread Herpes to their spouse or girlfriend or boyfriend. Herpes can (unfortunately) cause emotional pain. Also, it is very sad to say that Herpes can hurt or kill a baby—sometimes when a mother is giving birth—and the mother has Herpes—the baby can contract. Herpes during the birth. Because the baby is so young—the baby cannot fight off the infection. I would like to fix these sad issues.

### SUMMARY

MY system will intentionally infect a person with. Herpes Simplex 1 1) and Herpes Simplex 2 2). The infection shall be controlled and regulated. My system will also generate antibodies usv-1 (HERPES 1) and uSV-2 (HERPES 2). My system will prevent people from getting HERPES I and HERPES 2 on their genitals and other sensitive areas, and prevent people from getting HERPES 1 and HERPES 2 on their mouths. My system will also prevent babies from getting infected with HERPES I during birth and prevent babies from getting infected with HERPES 2 during birth.

Till/PINBALL HERPES ANTIBODY SYSTEM (THE SYSTEM) is a unique, complicated, time consuming, and expensive process that generates matter (HERPES I antibodies and HERPES 2 antibodies).

THE PINBALL HERPES ANTIBODY SYSTEM will not help people who are already infected with HERPES I and HERPES 2. someone is only with HERPES I then they could benefit from THE SYSTEM—they could be protected from HERPES 2. If someone is only infected with HERPES 2 Then they could benefit from THE SYSTEM They could be protected from HERPES 1.

THE PINBALL HERPES ANTIBODY SYSTEM is the only SYSTEM on the planet Earth that does these things.

THE PINBALL HERPES ANTIBODY SYSTEM will benefit humanity, society, and civilization. It will prevent millions of people, over thousands of years from getting terrible and painful sores on their private areas. And it will save babies THANK YOU.

### DESCRIPTION

THE PINBALL HERPES ANTIBODY SYSTEM (hereafter referred to as “THE SYSTEM”) is a highly regulated and controlled process. THE SYSTEM is designed to protect the PATIENT (described below) from the diseases of HERPES SIMPLEX

$$1 \begin{matrix}( HSV - 1 ) … ( A . K . A . HI \\ : 2 ( HSV - 2 ) … ( A . K . A .\end{matrix}$$

HERPES 1) and/or HERPES SIMPLEX 2 HERPES 2). And also to protect a baby during birth from getting HERPES.

THE SYSTEM is unique, complicated, involved, time consuming, and expensive.

### The Medical Clinic

THE SYSTEM shall consist of a professional medical clinic (hereafter referred to as “the clinic”). The clinic shall be under the direct supervision of one or more licensed Medical Doctor(s). The doctor(s) shall have both training and experience in dealing with the disease(s) of HERPES

$$\begin{matrix}( HSV - 1 ) … ( A . K . A . \\ ( HSV - 2 ) … ( A . K . A .\end{matrix}$$

SIMPLEX 1 HERPES 1) and HERPES SIMPLEX 2 HERPES 2). The clinic will also employ a professional HUMAN RESOURCE worker—to handle recruitment, direct control, and supervision of the HOST—(the HOST will be described below).

The medical clinic shall have the capacity to take blood samples from the HOSTS and the PATIENTS A licensed nurse(s) shall obtain blood samples from the HOST and the PATIENT—(the PATIENT shall be described below).

### The Patient

There will be two types of people who will utilize the clinic. One type of person will be the person who is currently not infected with HERPES I and/or not infected with HERPES 2. This type of person will be known as “The IENT”. The PATIENT is paying the clinic to provide The PATIENT with the PINBALL HERPES ANTI-BODY SYSTEM. The other person who will utilize the clinic will be a person who is already infected with HERPES I (I ISV1) or HERPES 2 (IISV-2) this already person shall be known as “the HOST”. The HOST is paid to provide HERPES I and HERPES 2 10 the clinic. The HOST will be subjected to extensive screening (Or their past behavior, and must pass a physical exam, and psychological exam, and health tests (as described below). The HOST is financially compensated (Or their time and effort. The HOST is treated with great respect by THE SYSTEM. Finding the correct HOST is a key part 10 THE SYSTF, Pvl it can be compared 0.10 finding the correct machine in order for a Factory 10 work properly (like a cog in the machinery). Finding the proper HOST could be very difficult and expensive. The HOST must have a certain background in order Ibr THE SYSTEM 10 work correctly. Everything must be put together just right—or THE PINBALL HERPES ANTIBODY SYSTEM will not work correctly.

The PATIENT will be intentionally infected with HERPES 1 and/or HERPES 2. The intentional HERPES infection shall be regulated and controlled (as described below). Licensed medical doctor(s) and licensed nurses shall handle and supervise the process.

### The Host

Selecting the correct HOST is a key part to the PINBALL HERPES ANTIBODY SYSTEM. The HOST will be providing the clinic with HERPES 1 and HERPES 2 samples. The HOST will be tested HERPES 1 and HERPES 2, and the AIDS virus. The HOST will be tested for the Hepatitis B virus. The HOST will be tested for the Hepatitis C virus. The HOST will be tested for Syphilis. The licensed nurse shall take the blood samples of the HOST at the clinic. The blood samples shall be subject to the latest, most reliable, and most sophisticated testing possible. The HUMAN RESOURCE worker will recruit the HOST. The HOST will be subject to questions regarding their personal behavior—such as illegal drug use and sexual activity. The HOST will be subject to psychological testing, screening, and background checks. The HOST shall be treated with the greatest respect—in a way the host will be a VERY IMPORTANT PERSON (V.I.P.). A good comparison could be having the correct quarterback on a football team—or else it does not matter if you have other good players on the team. [00191 The reason the HOST is subject to these tests and procedures—is that the HOST will be providing the HERPES samples from visible HERPES sores on the HOST'S lips. The transfer shall take place with a swab (as described below). The HOST must be in the proper physical condition and proper psychological condition to provide the PATIENT with HERPES 1 and/or HERPES 2. This is done to insure the PATIENT does not become infected with AIDS, nor Hepatitis B, nor Hepatitis C, nor Syphilis. The HOST will be subjected to psychological testing and screening. THE SYSTEM will not work correctly if the HOST does things—such as: injecting heroin the night before they are scheduled to contribute a HERPES sample and then exposing the IENT to AIDS or IIF, PAVITIS B. This is why it is so important to have the professional HUMAN RESOURCE worker as part of Till/PINBALL HERPES ANTIBODY SYSTEM. This part will take considerable time, effort, and money. The HUMAN RESOURCE worker will be responsible [Or the HOST to keep the HOST from doing the wrong things.

Some HOSTS will provide only HERPES 1 samples. Some HOSTS will provide only HERPES 2 samples. Some HOSTS will provide both HERPES 1 samples and HERPES 2 samples. The HUMAN RESOURCE worker and the DOCTOR(S) will analyze all relevant data to insure a potential HOST has all of the necessary Factors to safely provide the clinic with HERPES 1 and/or HERPES 2. If this section of THE SYSTEM is not done correctly a patient could become infected with AIDS or HEPATITIS B or HEPATITIS C or Syphilis. The best and most sophisticated and most reliable medical tests will be used 10 determine if the HOST is infected with HERPES=I and/or HERPES 2 and if they have a current HERPES 1 or HERPES 2 outbreak on their lips.

When a person (the PATIENT) wants 10 gain protection from HERPES I and/or HERPES 2 from infecting their genitals, mouth, anus, hands, face, and eyes—they can make an appointment with the clinic. At the clinic they will be subject to blood tests. The blood tests will be the best, most sophisticated, and most reliable possible. It will be determined if they are already infected with HERPES I or HERPES 2. If they are already infected with HERPES I they will not need THE SYSTEM for HERPES 1. But they could still use THE SYSTEM to get protected from HERPES 2. If they are already infected with HERPES 2 they will not need THE SYSTEM for HERPES 2. But they could still use THE SYSTEM to get protected from HERPES 1.

Once a person is infected with HERPES I—that person will develop HERPES I antibodies. Once a person has HERPES 1 antibodies they can no longer get HERPES 1. (But they could still have outbreaks at the original HERPES 1 infection area).

Once a person is infected with HERPES 2—that person will develop HERPES 2 antibodies. Once a person has HERPES 2 antibodies they can no longer get HERPES 2. (But they could still have outbreaks at the original HERPES 2 infection area).

If the PATIENT is not infected with HERPES 1 nor infected with HERPES 2: then THE PINBALL HERPES ANTIBODY SYSTEM will prevent the PATIENT from getting HERPES 1 and HERPES 2 on their genitals. And prevent the PATIENT from getting both HERPES 1 and HERPES 2 on or in their mouth. And prevent the PATIENT from getting both HERPES 1 and HERPES 2 on and inside their anus, also protecting their hands, face, and eyes. [00251 If the PATIENT is already infected with HERPES 1—then THE SYSTEM will not protect them from HERPES 1. But if the PATIENT is not infected with HERPES 2—then THE SYSTEM will protect them from HERPES 2.

If the PATIENT is already infected with HERPES 2—then THE SYSTEM will not protect them from HERPES 2. But if the PATIENT is not infected with HERPES I—then THE SYSTEM will protect them from HERPES 1.

Blood tests at the clinic will determine if the PATIENT has already been infected with HERPES 1 and/or HERPES 2. If the PATIENT has already been with both HERPES 1 and HERPES 2 then THE SYSTEM cannot help them.

If after the original blood tests it is determined that a PATIENT can benefit from Till/SYSTEM: then the PATIENT is subject to the next step in the process.

The PATIENT will be informed of the next step in Till/SYSTEM. The PATIENT will be intentionally infected with: HERPES II only; or HERPES 2 only; or both HERPES 1 and HERPES 2. The PATIENT will decide on what type of infection they will get after discussing the situation with the doctor(s).

The intentional infection process will take place at a location or locations on the PATIENT'S body that will not be as sensitive as their genitals, nor mouth, nor anus (which are normal areas Ibr HERPES infection).

### The Swabbing

The intentional HERPES infection shall be at a location on the PATIENT'S body the PATIENT has selected 10 be intentionally infected. The PATIENT shall discuss the situation with the doctor(s) And then make a decision. If the PATIENT is only going 10 gel one intentional HERPES infection then only one location will be selected by the PATIENT. If the PATIENT is 10 gel two intentional HERPES infections—then the PATIENT shall select two areas on their body to be infected. The PATIENT shall have full authority on where the intentional HERPES infection shall be. If the PATIENT is intentionally getting infected with both HERPES 1 and HERPES 2 the PATIENT will make the decision for both locations. Possible non-sensitive locations could be under the upper arm—by the armpit or on the top of a foot. Or some other area of the body—as determined by the PATIENT (after discussing the situation with the doctor(s)).

The area(s) of intentional HERPES infection shall be subjected to a medically professional incision made by the doctor(s) at the clinic. The equipment used for the incision shall be medically professional and medically sterile. The intentional infection shall be done by the doctor(s) or licensed nurse—using a swab. The doctor(s) shall determine the best material possible to make and use for the swab. The swab shall be medically professionally made and medically professionally disinfected and medically professionally clean—and kept in a medically professional sterile package. And kept in the medically professional sterile package until ready for immediate use.

The doctor(s) or licensed nurse shall take the sterile swab and use the sterile swab to make contact with the HOST'S visible HERPES 1 sore(s) on the HOST'S lips. And then intentionally transfer the HERPES I virus from the HOST'S lips to the PATIENT'S intentional incision for the HERPES I intentional infection.

The doctor(s) or licensed nurse shall take the sterile swab and use the sterile swab to make contact with the HOST'S visible HERPES 2 sore(s) on the HOST'S lips. And then intentionally transfer the HERPES 2 virus from the HOST'S lips to the PATIENT'S intentional incision for the HERPES 2 intentional infection.

If the PATIENT is only getting infected with HERPES I—then only one incision will be made. If the PATIENT is only getting infected with HERPES 2—then only one incision will be made. If the PATIENT is getting infected with both HERPES 1 and HERPES 2—then the doctor(s) will make two incisions. This is done to see if and when an original HERPES outbreak has occurred. One location will be (Or HERPES I and the other location will be for HERPES 2. The doctor(s) will keep a record of where each and what type of intentional infection took place. This will help the doctor(s) determine if an infection actually took place (as described below)—or whether the intentional infection process (the swabbing process) needs to be repeated. And if it needs to be repeated the doctor(s) will know which infection needs to be I or HERPES 2). Also, sophisticated, expensive, and reliable blood tests will be done about 21 to 42 days after the original outbreak both HERPES 1 and HERPES 2 to determine if HERPES antibodies have been generated by the PATIENT'S body. And exactly which HERPES antibodies have been generated (HERPES I or HERPES 2 or both).

### Infection

A typical HERPES I infection will occur from 2 days 10 21 days after original infection. After 21 days the doctor(s) will conduct the most reliable, most modern, sophisticated, and expensive blood test(s) to determine if any and what type of HERPES infection(s) look place. If the IENT does not have a visible HERPES outbreak alter 21 days then it is possible that the PATIENT will not have a visible outbreak. But, the patient could still have been infected (meaning the intentional swabbing was effective). The blood tests will provide the doctor(s) with the information needed to determine whether or not there was a HERPES I infection or a HERPES 2 infection or no infection at all. The doctor(s) will then wait another 21 to 42 days to conduct new sophisticated, most reliable, most modern, and expensive blood tests to determine whether or not HERPES I antibodies have been generated by the PATIENT.

If HERPES I antibodies have been detected (by the blood est(s)—then THE SYSTEM worked for HERPES 1. 100381 If HERPES I antibodies have not been detected—then the swabbing did not generate a HERPES I infection. The process is repeated for HERPES 1.

Once the PATIENT has antibodies for HERPES I—then the PATIENT is immuned to getting a HERPES 1 infection anywhere else on their body.

Once antibodies for HERPES I are generated by a person's body—the antibodies for HERPES I are generated for the life of that person.

A typical HERPES 2 infection will occur from 2 days to 21 days after original infection. After 21 days the doctor(s) will conduct the most reliable, most modern, expensive, and sophisticated blood test(s) to determine if any and what type of HERPES infection(s) took place. If the PATIENT does not have a visible HERPES outbreak after 21 days—then it is possible that the PATIENT will not have a visible outbreak. But, the patient could still have been infected (meaning the intentional swabbing was effective). The blood tests will provide the doctors with the information needed to determine whether or not there was a HERPES I infection or a HERPES 2 infection or no infection at all. The doctor(s) will then wait another 21 to 42 days to conduct new sophisticated, most reliable, most modern, and expensive blood tests to determine whether or not HERPES 2 antibodies have been generated by the PATIENT.

If HERPES 2 antibodies have been detected (by the blood est(s))—then THE SYSTEM worked for HERPES 2. 100431 If HERPES 2 antibodies have not been detected then the swabbing did not generate a HERPES 2 infection. The process is repeated (Or HERPES 2.

Once the PATIENT has antibodies for HERPES 2_then the PATIENT is immuned to getting a HERPES 2 infection anywhere else on their body.

Once antibodies for HERPES 2 are generated by a person's body the antibodies [Or HERPES 2 are generated for the life of that person.

### Protection [Or a Baby

If a mother has generated antibodies (Or both HERPES 1 and HERPES 2 with the PINBALL HERPES ANTIBODY SYSTEM: then the mother does not need to worry about getting HERPES 1 nor HERPES 2. so there is now no chance of her baby getting HERPES during birth.

### Flowchart for the Pinball Herpes Antibody System

ESTABLISH MEDICAL CLINIC
RECRUIT THE HOST
CONDUCT BLOOD TESTS and PSYCHOLOGICAL SCREENING ON HOST
GET PATIENT—START PROCESS
CONDUCT BLOOD TESTS ON PATIENT
PATIENT DECIDES WHAT ANTIBODIES DO THEY WANT (HERPES 1 or HERPES 2 or BOTH)
PATIENT DECIDES WHERE TO BE INFECTED
INTENTIONALLY INFECT PATIENT
WAIT 2 to 21 DAYS for INFECTION TO GENERATE (OUTBREAK)
BLOOD TESTS TO DETERMINE IF and WHAT TYPE OF INFECTION TOOK PLACE
WAIT 21 to 42 DAYS AFTER OUTBREAK
TEST PATIENT FOR ANTIBODIES
PATIENT IMMUNED or REPEAT PROCESS

1. A system for producing an immune response to Herpes-1 in humans, comprising the steps of:
recruiting prospective hosts, who are otherwise healthy persons who have already been infected with Herpes-1;
screening the prospective hosts for communicable blood transferred pathogens;
selecting hosts from those who have passed the screening;
taking virus particles from the hosts;
making incisions on intentional infection locations on the bodies of patients;
putting enough virus particles into the incisions to cause an immune response, but not so many as to produce painful sores.

2. The system for producing an immune response to Herpes-1 in humans according to claim 1, wherein:
the communicable blood transferred pathogens that are screened for include those that cause AIDS, Hepatitis, and syphilis.

3. The system for producing an immune response to Herpes-1 in humans according to claim 1, wherein:
the number of virus particles put into each incision shall be greater than ten thousand.

4. The system for producing an immune response to Herpes-1 in humans according to claim 1, wherein:
the intentional infection locations shall be on the upper inside back of one of each patient's leg.

5. The system for producing an immune response to Herpes-1 in humans according to claim 1, wherein:
the incisions are made by trained medical personnel.

6. The system for producing an immune response to Herpes-1 in humans according to claim 1, wherein:
after the virus particles are taken from the hosts,
they are placed in a medically professional storage system,
before they are placed into the incisions in the patients.

7. The system for producing an immune response to Herpes-1 in humans according to claim 1, wherein:
the system generates matter, namely, Herpes Simplex Virus 1 antibodies.

8. A system for producing immunity to Herpes-1 in humans, comprising the steps of:
recruiting prospective hosts, who are otherwise healthy persons who have already been infected with Herpes-1;
screening the prospective hosts for communicable blood transferred pathogens;
selecting hosts from those who have passed the screening;
taking virus particles from the hosts;
making incisions on intentional infection locations on the bodies of patients;
putting enough virus particles into the incisions to cause a sufficient immune response to create immunity to Herpes-1 in the patients.

9. The system for producing immunity to Herpes-1 in humans according to claim 8, wherein:
the communicable blood transferred pathogens that are screened for include those that cause AIDS, Hepatitis, and syphilis.

10. The system for producing immunity to Herpes-1 in humans according to claim 8, wherein:
the number of virus particles put into each incision shall be greater than ten thousand.

11. The system for producing immunity to Herpes-1 in humans according to claim 8, wherein:
the intentional infection locations shall be on the upper inside back of one of each patient's leg.

12. The system for producing immunity to Herpes-1 in humans according to claim 8, wherein:
the incisions are made by trained medical personnel.

13. The system for producing immunity to Herpes-1 in humans according to claim 8, wherein:
after the virus particles are taken from the hosts,
they are placed in a medically professional storage system,
before they are placed into the incisions in the patients.

14. The system for producing immunity to Herpes-1 in humans according to claim 8, wherein:
the system generates matter, namely, Herpes Simplex Virus 1 antibodies.

15. A method for producing a composition of matter, namely, antibodies to Herpes-1, comprising the steps of:
recruiting prospective hosts, who are otherwise healthy persons who have already been infected with Herpes-1;
screening the prospective hosts for communicable blood transferred pathogens;
selecting hosts from those who have passed the screening;
taking virus particles from the hosts;
making incisions on intentional infection locations on the bodies of patients;
putting enough virus particles into the incisions to cause a sufficient immune response to create immunity to Herpes-1 in the patients; and
the system generates matter, namely, Herpes Simplex Virus 1 antibodies.

16. The system for producing immunity to Herpes-1 in humans according to claim 15, wherein:
the communicable blood transferred pathogens that are screened for include those that cause AIDS, Hepatitis, and syphilis.

17. The system for producing immunity to Herpes-1 in humans according to claim 15, wherein:
the number of virus particles put into each incision shall be greater than ten thousand.

18. The system for producing immunity to Herpes-1 in humans according to claim 15, wherein:
the intentional infection locations shall be on the upper inside back of one of each patient's leg.

19. The system for producing immunity to Herpes-1 in humans according to claim 15, wherein:
the incisions are made by trained medical personnel.

20. The system for producing immunity to Herpes-1 in humans according to claim 15, wherein:
after the virus particles are taken from the hosts,
they are placed in a medically professional storage system,
before they are placed into the incisions in the patients.
